SEQUENCE LISTING (35 sequences)

SEQ ID NO: 1 - DNA, 519 bp, synthetic construct
  misc_feature 1..519: note = Synthetic cDNA encoding Der p2 natural form
  source 1..519: mol_type = other DNA
tctagaggtaccatgaagaccaacctgttcttgttcctgatcttcagcctgctgctgagcctgtcatctgctgatcaagtggatgtgaaggattgcgctaaccacgagatcaagaaggtgttggttcctggttgccatggttctgagccttgcattattcacaggggtaagcctttccagcttgaggctgttttcgaggctaaccagaataccaagaccgctaagattgagatcaaggctagcatcgatggtctggaagttgatgtgcctggtatcgatcctaacgcttgccactatatgaagtgccctcttgtgaagggtcagcagtacgatatcaagtacacctggaacgtgccaaagatcgctcctaagtctgagaatgtggtggtgaccgttaaggtgatgggtgatgatggtgttctggcttgcgctattgctacccatgctaagattagggatcatcaccaccaccatcacgattacaaggacgatgatgataaagtgcctaggtaagtcgac

SEQ ID NO: 2 - protein, 166 aa, Dermatophagoides pteronyssinus
MKTNLFLFLIFSLLLSLSSADQVDVKDCANHEIKKVLVPGCHGSEPCIIHRGKPFQLEAVFEANQNTKTAKIEIKASIDGLEVDVPGIDPNACHYMKCPLVKGQQYDIKYTWNVPKIAPKSENVVVTVKVMGDDGVLACAIATHAKIRDHHHHHHDYKDDDDKVPR

SEQ ID NO: 3 - DNA, 695 bp, synthetic construct
  misc_feature 1..695: note = Synthetic cDNA according to invention B
  source 1..695: mol_type = other DNA
tctagaggtaccatgaagaccaacctgttcttgttcctgatcttcagcctgctgctgagcctgtcatctgctgatcaagtggatgtgaaggattgcgctaaccacgagatcaagaaggtgttggttcctggttgccatggttctgagccttgcattattcacaggggtaagcctttccagcttgaggctgttttcgaggctaaccagaataccaagaccgctaagattgagatcaaggctagcatcgatggtctggaagttgatgtgcctggtatcgatcctaacgcttgccactatatgaagtgccctcttgtgaagggtcagcagtacgatatcaagtacacctggaacgtgccaaagatcgctcctaagtctgagaatgtggtggtgaccgttaaggtgatgggtgatgatggtgttctggcttgcgctattgctacccatgctaagattagggatggtggtggttctggtggtggatctcttaagcagattgaggataagatcgaagagatcctgagcaagatctaccacatcgagaacgagatcgctaggatcaagaagctgatcggagaatctgctgctggtggtggtagttaccagatcctgtctatctacagcaccgtggcttcatctcttgctctggctattatgatggctggtctgtctctgtggatgtgctctaacggttctct

SEQ ID NO: 4 - protein, 234 aa, synthetic construct
  REGION 1..234: note = Synthetic protein according to invention B
MKTNLFLFLIFSLLLSLSSADQVDVKDCANHEIKKVLVPGCHGSEPCIIHRGKPFQLEAVFEANQNTKTAKIEIKASIDGLEVDVPGIDPNACHYMKCPLVKGQQYDIKYTWNVPKIAPKSENVVVTVKVMGDDGVLACAIATHAKIRDGGGSGGGSLKQIEDKIEEILSKIYHIENEIARIKKLIGESAAGGGSYQILSIYSTVASSLALAIMMAGLSLWMCSNGSLQCRICI

SEQ ID NO: 5 - DNA, 726 bp, synthetic construct
  misc_feature 1..726: note = Synthetic cDNA according to invention C
  source 1..726: mol_type = other DNA
tctagaggtaccatgaagaccaacctgttcttgttcctgatcttcagcctgctgctgagcctgtcatctgctgatcaagtggatgtgaaggattgcgctaaccacgagatcaagaaggtgttggttcctggttgccatggttctgagccttgcattattcacaggggtaagcctttccagcttgaggctgttttcgaggctaaccagaataccaagaccgctaagattgagatcaaggctagcatcgatggtctggaagttgatgtgcctggtatcgatcctaacgcttgccactatatgaagtgccctcttgtgaagggtcagcagtacgatatcaagtacacctggaacgtgccaaagatcgctcctaagtctgagaatgtggtggtgaccgttaaggtgatgggtgatgatggtgttctggcttgcgctattgctacccatgctaagattagggatggtggtggttctggtggtggatctaggatgaagcagatcgaggataagctggaagagatcctgagcaagctgtaccacattgagaacgagctggctaggatcaagaagctgcttggagaaaggggtggtggtagttaccagatcctgtccatctactctaccgtggcttcttctcttgctctggctatcatgatggctggtctgtctctttggatgtgcagcaacggttctcttcagtgcaggatctgcatctaaactagtgtcgac

SEQ ID NO: 6 - protein, 233 aa, synthetic construct
  REGION 1..233: note = Synthetic Protein according to invention C
MKTNLFLFLIFSLLLSLSSADQVDVKDCANHEIKKVLVPGCHGSEPCIIHRGKPFQLEAVFEANQNTKTAKIEIKASIDGLEVDVPGIDPNACHYMKCPLVKGQQYDIKYTWNVPKIAPKSENVVVTVKVMGDDGVLACAIATHAKIRDGGGSGGGSRMKQIEDKLEEILSKLYHIENELARIKKLLGERGGGSYQILSIYSTVASSLALAIMMAGLSLWMCSNGSLQCRICI

SEQ ID NO: 7 - DNA, 723 bp, synthetic construct
  misc_feature 1..723: note = Synthetic cDNA according to invention D
  source 1..723: mol_type = other DNA
tctagaggtaccatgaagaccaacctgttcttgttcctgatcttcagcctgctgctgagcctgtcatctgctgatcaagtggatgtgaaggattgcgctaaccacgagatcaagaaggtgttggttcctggttgccatggttctgagccttgcattattcacaggggtaagcctttccagcttgaggctgttttcgaggctaaccagaataccaagaccgctaagattgagatcaaggctagcatcgatggtctggaagttgatgtgcctggtatcgatcctaacgcttgccactatatgaagtgccctcttgtgaagggtcagcagtacgatatcaagtacacctggaacgtgccaaagatcgctcctaagtctgagaatgtggtggtgaccgttaaggtgatgggtgatgatggtgttctggcttgcgctattgctacccatgctaagattagggatggtggtggttctggtggtggatctgtgaagcagcttgctgatgctgttgaggaactggcttctgctaactaccaccttgctaacgctgttgctaggttggctaaggctgttggtgaaaggggtggtggtagttaccagatcctgtccatctactctaccgtggcttcttctcttgctctggctatcatgatggctggtctgtctctttggatgtgcagcaacggttctcttcagtgcaggatctgcatctaaactagtgtcgac

SEQ ID NO: 8 - protein, 232 aa, synthetic construct
  REGION 1..232: note = Synthetic Protein according to invention D
MKTNLFLFLIFSLLLSLSSADQVDVKDCANHEIKKVLVPGCHGSEPCIIHRGKPFQLEAVFEANQNTKTAKIEIKASIDGLEVDVPGIDPNACHYMKCPLVKGQQYDIKYTWNVPKIAPKSENVVVTVKVMGDDGVLACAIATHAKIRDGGGSGGGSVKQLADAVEELASANYHLANAVARLAKAVGERGGGSYQILSIYSTVASSLALAIMMAGLSLWMCSNGSLQCRICI

SEQ ID NO: 9 - DNA, 723 bp, synthetic construct
  misc_feature 1..723: note = Synthetic cDNA according to invention E
  source 1..723: mol_type = other DNA
tctagaggtaccatgaagaccaacctgttcttgttcctgatcttcagcctgctgctgagcctgtcatctgctgatcaagtggatgtgaaggattgcgctaaccacgagatcaagaaggtgttggttcctggttgccatggttctgagccttgcattattcacaggggtaagcctttccagcttgaggctgttttcgaggctaaccagaataccaagaccgctaagattgagatcaaggctagcatcgatggtctggaagttgatgtgcctggtatcgatcctaacgcttgccactatatgaagtgccctcttgtgaagggtcagcagtacgatatcaagtacacctggaacgtgccaaagatcgctcctaagtctgagaatgtggtggtgaccgttaaggtgatgggtgatgatggtgttctggcttgcgctattgctacccatgctaagattagggatggtggtggttctggtggtggatctaagcagatcgaggataagatcgagaacatcaccagcaagatctacaacattaccaacgagatcgctaggatcaagaagctgatcggtaacaggaccggtggtggtagttaccagatcctgtccatctactctaccgtggcttcttctcttgctctggctatcatgatggctggtctgtctctttggatgtgcagcaacggttctcttcagtgcaggatctgcatctaaactagtgtcgac

SEQ ID NO: 10 - protein, 232 aa, synthetic construct
  REGION 1..232: note = Synthetic Protein according to invention E
MKTNLFLFLIFSLLLSLSSADQVDVKDCANHEIKKVLVPGCHGSEPCIIHRGKPFQLEAVFEANQNTKTAKIEIKASIDGLEVDVPGIDPNACHYMKCPLVKGQQYDIKYTWNVPKIAPKSENVVVTVKVMGDDGVLACAIATHAKIRDGGGSGGGSKQIEDKIENITSKIYNITNEIARIKKLIGNRTGGGSYQILSIYSTVASSLALAIMMAGLSLWMCSNGSLQCRICI

SEQ ID NO: 11 - DNA, 705 bp, synthetic construct
  misc_feature 1..705: note = Synthetic cDNA according to invention F
  source 1..705: mol_type = other DNA
tctagaggtaccatgaagaccaacctgttcttgttcctgatcttcagcctgctgctgagcctgtcatctgctgatcaagtggatgtgaaggattgcgctaaccacgagatcaagaaggtgttggttcctggttgccatggttctgagccttgcattattcacaggggtaagcctttccagcttgaggctgttttcgaggctaaccagaataccaagaccgctaagattgagatcaaggctagcatcgatggtctggaagttgatgtgcctggtatcgatcctaacgcttgccactatatgaagtgccctcttgtgaagggtcagcagtacgatatcaagtacacctggaacgtgccaaagatcgctcctaagtctgagaatgtggtggtgaccgttaaggtgatgggtgatgatggtgttctggcttgcgctattgctacccatgctaagattagggatggtggtggttctggtggtggatctctgaagtccaggcttgataccctgtctcaagaggtggcacttctgaaagaacagcaggctctgcagactgtgtgtctgggtggtggtagttaccagatcctgtccatctactctaccgtggcttcttctcttgctctggctatcatgatggctggtctgtctctttggatgtgcagcaacggttctcttcagtgcaggatctgcatctaaactagtgtcgac

SEQ ID NO: 12 - protein, 226 aa, synthetic construct
  REGION 1..226: note = Synthetic Protein according to invention F
MKTNLFLFLIFSLLLSLSSADQVDVKDCANHEIKKVLVPGCHGSEPCIIHRGKPFQLEAVFEANQNTKTAKIEIKASIDGLEVDVPGIDPNACHYMKCPLVKGQQYDIKYTWNVPKIAPKSENVVVTVKVMGDDGVLACAIATHAKIRDGGGSGGGSLKSRLDTLSQEVALLKEQQALQTVCLGGGSYQILSIYSTVASSLALAIMMAGLSLWMCSNGSLQCRICI

SEQ ID NO: 13 - DNA, 705 bp, synthetic construct
  misc_feature 1..705: note = Synthetic cDNA according to invention G
  source 1..705: mol_type = other DNA
tctagaggtaccatgaagaccaacctgttcttgttcctgatcttcagcctgctgctgagcctgtcatctgctgatcaagtggatgtgaaggattgcgctaaccacgagatcaagaaggtgttggttcctggttgccatggttctgagccttgcattattcacaggggtaagcctttccagcttgaggctgttttcgaggctaaccagaataccaagaccgctaagattgagatcaaggctagcatcgatggtctggaagttgatgtgcctggtatcgatcctaacgcttgccactatatgaagtgccctcttgtgaagggtcagcagtacgatatcaagtacacctggaacgtgccaaagatcgctcctaagtctgagaatgtggtggtgaccgttaaggtgatgggtgatgatggtgttctggcttgcgctattgctacccatgctaagattagggatggtggtggttctggtggtggatctatcaacgagactgctgatgatatcgtgtacaggctgaccgtgattatcgatgataggtacgagagcctgaagaaccttggtggtggtagttaccagatcctgtccatctactctaccgtggcttcttctcttgctctggctatcatgatggctggtctgtctctttggatgtgcagcaacggttctcttcagtgcaggatctgcatctaaactagtgtcgac

SEQ ID NO: 14 - protein, 226 aa, synthetic construct
  REGION 1..226: note = Synthetic Protein according to invention G
MKTNLFLFLIFSLLLSLSSADQVDVKDCANHEIKKVLVPGCHGSEPCIIHRGKPFQLEAVFEANQNTKTAKIEIKASIDGLEVDVPGIDPNACHYMKCPLVKGQQYDIKYTWNVPKIAPKSENVVVTVKVMGDDGVLACAIATHAKIRDGGGSGGGSINETADDIVYRLTVIIDDRYESLKNLGGGSYQILSIYSTVASSLALAIMMAGLSLWMCSNGSLQCRICI

SEQ ID NO: 15 - DNA, 573 bp, synthetic construct
  misc_feature 1..573: note = Synthetic cDNA according to invention H
  source 1..573: mol_type = other DNA
tctagaggtaccatgaagaccaacctgttcttgttcctgatcttcagcctgctgctgagcctgtcatctgcttgccatggttctgagccttgcattattcacaggggtaagcctttccagcttgaggctgttttcgaggctaaccagaataccaagaccgctaagggtggtggtagtgaagttgatgtgcctggtatcgatcctaacgcttgccactatatgaagtgccctcttgtgaagggtcagcagtacgatatcaagtacacctggaacgtgccaaagatcgctcctaagtctgagaatggtggtggatctggtggtggatcccttaagcagattgaggataagatcgaagagatcctgagcaagatctaccacatcgagaacgagatcgctaggatcaagaagctgatcggagaatctgctgctggtggtggtagttaccagatcctgtccatctactctaccgtggcttcttctcttgctctggctatcatgatggctggtctgtctctttggatgtgcagcaacggttctcttcagtgcaggatctgcatctaaactagtgtcgac

SEQ ID NO: 16 - protein, 182 aa, synthetic construct
  REGION 1..182: note = Synthetic Protein according to invention H
MKTNLFLFLIFSLLLSLSSACHGSEPCIIHRGKPFQLEAVFEANQNTKTAKGGGSEVDVPGIDPNACHYMKCPLVKGQQYDIKYTWNVPKIAPKSENGGGSGGGSLKQIEDKIEEILSKIYHIENEIARIKKLIGESAAGGGSYQILSIYSTVASSLALAIMMAGLSLWMCSNGSLQCRICI

SEQ ID NO: 17 - DNA, 579 bp, synthetic construct
  misc_feature 1..579: note = Synthetic cDNA according to invention I
  source 1..579: mol_type = other DNA
tctagaggtaccatgaagaccaacctgttcttgttcctgatcttcagcctgctgctgagcctgtcatctgctgagatctgcccagctgttaagagggacgtcgatctatttcttacgggaactcctgatgagtacgtcgaacaagttgcacagtacaaagcactaccagtagtactcgagaacgctaggatacttaagaattgtgtggacgcaaaaatgaccgaggaagacaaggaaaacgcattgagcttgcttgataaaatatacacctctccactttgccatcaccaccaccatcacactagtggtggtggttctggtggtggatcccttaagcagattgaggataagatcgaagagatcctgagcaagatctaccacatcgagaacgagatcgctaggatcaagaagctgatcggagaatctgctgctggtggtggtagttaccagatcctgtctatctacagcaccgtggcttcatctcttgctctggctattatgatggctggtctgtctctgtggatgtgctctaacggttctcttcagtgcaggatctgcatttaaactagttaagtcgac

SEQ ID NO: 18 - protein, 183 aa, synthetic construct
  REGION 1..183: note = Synthetic Protein according to invention I
MKTNLFLFLIFSLLLSLSSAEICPAVKRDVDLFLTGTPDEYVEQVAQYKALPVVLENARILKNCVDAKMTEEDKENALSLLDKIYTSPLCHHHHHHTSGGGSGGGSLKQIEDKIEEILSKIYHIENEIARIKKLIGESAAGGGSYQILSIYSTVASSLALAIMMAGLSLWMCSNGSLQCRICI

SEQ ID NO: 19 - DNA, 709 bp, synthetic construct
  misc_feature 1..709: note = Synthetic cDNA according to invention J
  source 1..709: mol_type = other DNA
tctagaggtaccatgaagaccaacctgttcttgttcctgatcttcagcctgctgctgagcctgtcatctgctgatcaagtggatgtgaaggattgcgctaaccacgagatcaagaaggtgttggttcctggttgccatggttctgagccttgcattattcacaggggtaagcctttccagcttgaggctgttttcgaggctaaccagaataccaagaccgctaagattgagatcaaggctagcatcgatggtctggaagttgatgtgcctggtatcgatcctaacgcttgccactatatgaagtgccctcttgtgaagggtcagcagtacgatatcaagtacacctggaacgtgccaaagatcgctcctaagtctgagaatgtggtggtgaccgttaaggtgatgggtgatgatggtgttctggcttgcgctattgctacccatgctaagattagggatggtggtggttctggtggtggatctcttaagcagattgaggataagatcgaagagatcctgagcaagatctaccacatcgagaacgagatcgctaggatcaagaagctgatcggagaatctgctgctggtggtggtagtattgctcttgctgttggtggtgttgctgtgctgggtttcgttattgtgtgccttctggtgctgaagtccgctatgaagaagaagtccaagtacgatagctactagtgtcgac

SEQ ID NO: 20 - protein, 229 aa, synthetic construct
  REGION 1..229: note = Synthetic Protein according to invention J
MKTNLFLFLIFSLLLSLSSADQVDVKDCANHEIKKVLVPGCHGSEPCIIHRGKPFQLEAVFEANQNTKTAKIEIKASIDGLEVDVPGIDPNACHYMKCPLVKGQQYDIKYTWNVPKIAPKSENVVVTVKVMGDDGVLACAIATHAKIRDGGGSGGGSLKQIEDKIEEILSKIYHIENEIARIKKLIGESAAGGGSIALAVGGVAVLGFVIVCLLVLKSAMKKKSKYDSY

SEQ ID NO: 21 - protein, 20 aa, synthetic construct
  REGION 1..20: note = Synthetic Signal peptide of the tobacco chitinase
MKTNLFLFLIFSLLLSLSSA

SEQ ID NO: 22 - protein, 129 aa, Dermatophagoides pteronyssinus
DQVDVKDCANHEIKKVLVPGCHGSEPCIIHRGKPFQLEAVFEANQNTKTAKIEIKASIDGLEVDVPGIDPNACHYMKCPLVKGQQYDIKYTWNVPKIAPKSENVVVTVKVMGDDGVLACAIATHAKIRD

SEQ ID NO: 23 - protein, 8 aa, synthetic construct
  REGION 1..8: note = Synthetic Linker
GGGSGGGS

SEQ ID NO: 24 - protein, 34 aa, synthetic construct
  REGION 1..34: note = Synthetic GCN4-pII coiled-coil domain
LKQIEDKIEEILSKIYHIENEIARIKKLIGESAA

SEQ ID NO: 25 - protein, 4 aa, synthetic construct
  REGION 1..4: note = Synthetic Linker
GGGS

SEQ ID NO: 26 - protein, 39 aa, synthetic construct
  REGION 1..39: note = Synthetic Anchoring sequence of the H5N1 influenza virus H5 hemagglutinin
YQILSIYSTVASSLALAIMMAGLSLWMCSNGSLQCRICI

SEQ ID NO: 27 - protein, 33 aa, synthetic construct
  REGION 1..33: note = Synthetic GCN4-pLI Coiled-coil domain
RMKQIEDKLEEILSKLYHIENELARIKKLLGER

SEQ ID NO: 28 - protein, 32 aa, synthetic construct
  REGION 1..32: note = Synthetic GCN4-pAA coiled-coil domain
VKQLADAVEELASANYHLANAVARLAKAVGER

SEQ ID NO: 29 - protein, 32 aa, synthetic construct
  REGION 1..32: note = Synthetic IZN4 coiled-coil domain
KQIEDKIENITSKIYNITNEIARIKKLIGNRT

SEQ ID NO: 30 - protein, 26 aa, synthetic construct
  REGION 1..26: note = Synthetic SNARE coiled-coil domain
INETADDIVYRLTVIIDDRYESLKNL

SEQ ID NO: 31 - protein, 34 aa, synthetic construct
  REGION 1..34: note = Synthetic Anchoring sequence of the PDLP1 protein
IALAVGGVAVLGFVIVCLLVLKSAMKKKSKYDSY

SEQ ID NO: 32 - protein, 78 aa, synthetic construct
  REGION 1..78: note = Synthetic Sequence of the CH1 chain of the feline allergen Fel d 1
EICPAVKRDVDLFLTGTPDEYVEQVAQYKALPVVLENARILKNCVDAKMTEEDKENALSLLDKIYTSPLCHHHHHHTS

SEQ ID NO: 33 - protein, 26 aa, synthetic construct
  REGION 1..26: note = Synthetic Coiled-coil domain
LKSRLDTLSQEVALLKEQQALQTVCL

SEQ ID NO: 34 - protein, 328 aa, Influenza A virus
QDLPGNDNSTATLCLGHHAVPNGTLVKTITDDQIEVTNATELVQSSSTGKICNNPHRILDGIDCTLIDALLGDPHCDVFQNETWDLFVERSKAFSNCYPYDVPDYASLRSLVASSGTLEFITEGFTWTGVTQNGGSNACKRGPGSGFFSRLNWLTKSGSTYPVLNVTMPNNDNFDKLYIWGIHHPSTNQEQTSLYVQASGRVTVSTRRSQQTIIPNIGSRPWVRGLSSRISIYWTIVKPGDVLVINSNGNLIAPRGYFKMRTGKSSIMRSDAPIDTCISECITPNGSIPNDKPFQNVNKITYGACPKYVKQNTLKLATGMRNVPEKQT

SEQ ID NO: 35 - protein, 500 aa, synthetic construct
  REGION 1..500: note = Synthetic Zika virus envelope protein
IRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFTCSKKMTGKSIQPENLEYRIMLSVHGSQHSGMIGYETDEDRAKVEVTPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLFSGHLKCRLKMDKLRLKGVSYSLCTAAFTFTKVPAETLHGTVTVEVQYAGTDGPCKIPVQMAVDMQTLTPVGRLITANPVITESTENSKMMLELDPPFGDSYIVIGVGDKKITHHWHRSGSTIGKAFEATVRGAKRMAVLGDTAWDFGSVGGVFNSLGKGIHQIFGAAFKSLFGGMSWFSQILIGTLLVWLGLNTKNGSISLTCLALGGVMIFLSTAVSA

(19) United States
(12) Patent Application Publication
(10) Pub. No.: US 2026/0034210 A1
(43) Pub. Date: Feb. 5, 2026

(54) PSEUDO-VIRAL PARTICLES AND USES OF SAME

(71) Applicant: ANGANY INC., Québec (CA)

(72) Inventors: Véronique GOMORD, Rouen (FR); Louis-Philippe VEZINA, Quebec (CA); Loïc FAYE, St Jacques Sur Darnetal (FR); Virginie CATALA, Saint-Marcel (FR); Anne-Catherine FITCHETTE, Pavilly (FR)

(21) Appl. No.: 19/245,634

(22) Filed: Jun. 23, 2025

Related U.S. Application Data
(63) Continuation of application No. 16/320,719.

(30) Foreign Application Priority Data
Jul. 29, 2016 (FR) 16 57424

(51) Int. Cl. A61K 39/35; A61K 39/00; C07K 14/005; C07K 14/435

(52) U.S. Cl. CPC A61K 39/35; C07K 14/005; C07K 14/43527; A61K 2039/5256; A61K 2039/5258; A61K 2039/577; C07K 14/43563; C07K 2319/02; C07K 2319/03; C07K 2319/40; C07K 2319/73; C07K 2319/735; C12N 2760/16122; C12N 2760/16123; C12N 2760/16134

(57) ABSTRACT

The present invention relates to a type I or II transmembrane fusion protein comprising, successively:
a) optionally, a signal peptide;
b) a protein or a peptide of interest;
c) a coiled-coil domain; and
d) a domain for anchoring in the plasma membrane, consisting of a transmembrane segment and a cytosolic segment.

It also relates to the virus-like particles obtained with this fusion protein.

### CROSS-REFERENCE TO RELATED APPLICATIONS

The patent application is a continuation of U.S. application Ser. No. 16/320,719 filed Jan. 25, 2019, which is a 371 of PCT/FR2017/052146 filed on Jul. 28, 2017, which claimed priority of FR1657424 filed on Jul. 29, 2016.

### SEQUENCE LISTING

This document incorporates by reference an electronic sequence listing xml file, which was electronically submitted along with this document. The xml file is named 13500099US2_seqlisting.xml, is 54058 bytes, and was created on Jun. 23, 2025.

### FIELD OF THE INVENTION

The present invention relates to a fusion protein comprising the following fragments, successively:
a) optionally, a signal peptide;
b) a protein or a peptide of interest;
c) a coiled-coil domain which does not originate from a virus; and
d) a domain for anchoring in the plasma membrane, consisting of a transmembrane segment and a cytosolic segment, preferably a domain for anchoring in a plasma membrane of which at least one portion is typical of lipid rafts.

It also relates to the virus-like particles (VLPs) obtained with such a fusion protein, said protein being anchored in the membrane thereof.

Although allergen immunotherapy was described by Noon and Freeman (1, 2) more than 100 years ago, very little progress has been made in terms of desensitization, with the exception of the method of administration of treatments, with treatments by injection having gradually been replaced with desensitization via the sublingual route. Thus, since January 2011, there has for example in France been, for grass pollen allergies, a desensitization in the form of a sublingual tablet. The arrival on the market of these desensitization tablets has contributed to reducing the invasiveness of allergen immunotherapy, but it has not increased the efficacy thereof. Indeed, it is still “natural” allergenic extracts, that are poorly concentrated in allergens and not very representative of the diversity of the allergens contained in the allergenic source, that are used for oral curative treatment.

However, over the course of the past decade, new strategies have been proposed for increasing the efficacy and reducing the duration of allergen immunotherapy treatments.

These desensitization strategies are based on the use:
of natural allergenic extracts modified to reduce the allergenicity thereof while at the same time preserving the immunogenicity thereof (Henmar et al.),
of native recombinant allergens or recombinant allergens modified to make them hypoallergenic (Valenta et al.),
of peptides corresponding to the epitopes of T-cell allergens, in free form or in the form of a fusion with carrier proteins (Larche M., Patel D. et al., Chen et al.),
of adjuvants, or else
of allergens fused to nanoparticles or to virus-like particles (Kundig et al., Bachmann M F, Jennings G T, Henmar et al.).

However, the strategies which make use of modified allergenic extracts remain of limited efficacy because of their poor representativeness of the diversity of the allergens contained in the source.

Modified or non-modified recombinant allergens, or peptides, are weakly immunogenic in soluble form in the absence of adjuvants. However, many adjuvants are poorly tolerated and, as with vaccines, the use thereof is not recommended in allergen immunotherapy.

The use of allergens fused to nanoparticles or to virus-like particles (VLPs) is on the other hand a particularly attractive strategy for allergen immunotherapy. Virus-like particles self-assemble from viral antigens. They contain no genetic material, and thus they are non-infectious and incapable of multiplying. On the other hand, they mimic the original structure of a virus, which allows them to be easily recognized by the immune system and to very efficiently activate the immunological memory. VLP-based vaccines against hepatitis B, papillomavirus infections or the flu (Garland et al., Paavonen et al., D'Aoust et al.) illustrate the vaccine efficacy of antigens when they are presented to the immune system at the surface of VLPs or of nanoparticles. Thus, VLPs have the potential to be used as structures presenting antigens and in particular allergens which make it possible to induce a strong immune response in human beings. There are two major types of VLPs: those which are produced from viral capsid proteins (CP VLPs) and those which are produced from enveloped viruses (Env VLPs). The structure and the composition of these two types differ substantially. CP VLPs are generally produced by producing the recombinant protein of the capsid which self-assembles in host cells according to mechanisms similar to those of native viruses. The production of Env VLPs occurs when an envelope protein Env is synthesized and modified by the endomembrane system of a host cell, then migrates to the lipid rafts of the plasma membrane, where it becomes concentrated and triggers the extracellular budding of the entire membrane/protein assembly. The resulting particle, the VLP, carries at its surface immunogenic epitopes of the Env protein.

The use of allergens in the form of virus-like particles thus appears to be an important requirement for successful immunotherapy. The results obtained with VLPs fused to a peptide of the major acarid allergen (Der p 1) or the major cat allergen (Fel d 1) have illustrated the very great immunogenicity of these fusions in mice and in human beings (Schmitz et al., Kundig et al.). This immunogenicity is so high that a single injection of these VLPs induces a sufficient IgG production for protection against a type I allergic reaction.

However, the preparation of these VLPs is unfortunately extremely complex and comprises numerous steps, some of which are difficult to standardize. In particular, the VLPs are produced, on the one hand, in E. coli, after expression of the Qbeta bacteriophage envelope protein. On the other hand, the allergen is expressed in recombinant form in E.coli, purified, then solubilized and purified again in several steps. Once these two constituents have been produced and purified, they are coupled in vitro. This production technique is obviously too complex, too expensive and too difficult to standardize for it to be possible for the final product to one day be available for the treatment of allergic patients.

There is therefore a need for a structure that is non-immunogenic as such and polyvalent, which self-assembles in eukaryotic cells, which is easy to synthesize, and which can carry proteins or peptides on its surface. Such a structure could be used for the treatment of allergic patients, but also in other clinical contexts.

The applicant has now developed such a structure, which self-assembles and allows use in therapy.

In particular, the unique characteristics of such a structure are:
an undetectable immunogenicity of the structure as such;
its ability to self-assemble into oligomers, such as trimers or tetramers;
its membrane containing unique lipids, typical of lipid rafts;
its membrane with a low content of host-cell membrane proteins;
its ability to be expressed at high yields in numerous eukaryotic cell types (yeasts, insects or plants); and
its ease of preparation.

According to a first aspect, the invention relates to a type I or type II transmembrane fusion protein comprising the following fragments, successively:
a) optionally, a signal peptide;
b) a protein or a peptide of interest;
c) a coiled-coil domain (or oligomerization sequence) which does not originate from a virus; and
d) a domain for anchoring in the plasma membrane and more particularly in the lipid rafts, consisting of a transmembrane segment and a cytosolic segment.

Such a fusion protein behaves like a viral surface protein when it is expressed in eukaryotic cells.

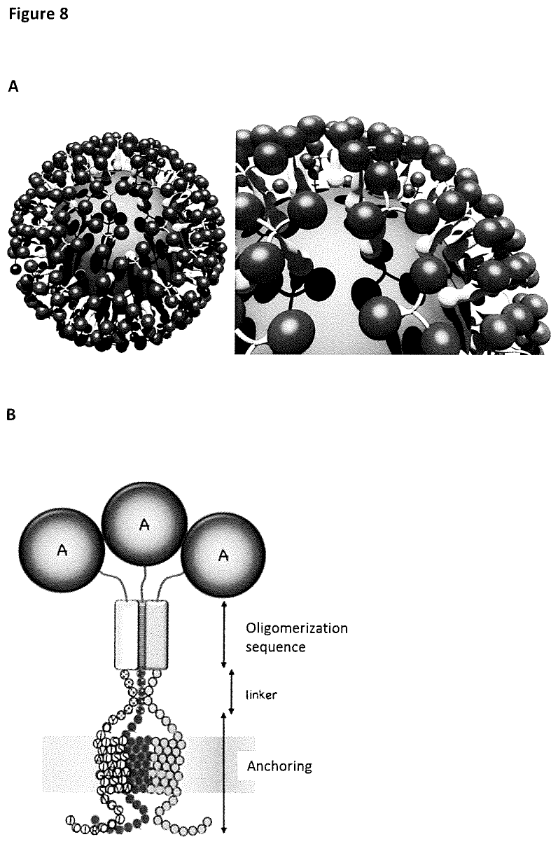

Without being bound by any theory, this protein is synthesized in the endoplasmic reticulum and then transported, via the Golgi apparatus, to the plasma membrane. Once it reaches specialized zones of the plasma membrane, preferably of the lipid rafts, this transmembrane fusion protein causes curving of said membrane, which finally forms a bud which separates from the cell membrane and is released into the extracellular space. During the budding, the protein or the peptide of interest carried by the coiled-coil domain (or oligomerization sequence) is exposed at the outer surface of the newly formed particle. The transmembrane domain remains anchored in the membrane and is not exposed at the surface. A virus-like particle (VLP), illustrated in FIG. 8A, is thus obtained, comprising a plasma membrane of which the composition is preferably typical of lipid rafts, in which the fusion proteins according to the invention are attached at the level of their anchoring domain and which exposes the protein or the peptide of interest at its surface, in oligomerized form (by virtue of the oligomerization sequence).

The structure of the fusion proteins assembled at the surface of the VIP is illustrated in FIG. 8B.

According to a second aspect, the invention consists of a virus-like particle (VLP) comprising:
an envelope consisting of a plasma membrane of which at least one portion is typical of lipid rafts; and
at least one type I or II transmembrane fusion protein anchored in said membrane (i.e. said envelope), said fusion protein comprising the following fragments, successively:
b) a protein or a peptide of interest;
c) a coiled-coil domain (or oligomerization sequence) which does not originate from a virus; and
d) a domain for anchoring in the plasma membrane, consisting of a transmembrane segment and a cytosolic segment, preferably a domain for anchoring in a plasma membrane of which at least one portion is typical of lipid rafts,
fragments b) and c) being exposed at the surface of the VLP.

In the VLP according to the invention, the fusion protein is anchored in the membrane (and thus in the envelope) by means of its anchoring domain d).

The term “virus-like particle (or VLP)” is intended to mean a nanoparticle consisting of a plasma membrane envelope in which one or more proteins are anchored, which contains no genetic material, which is non-infectious and incapable of multiplying, and which self-assembles to mimic the original structure of a virus. The structure of a VLP is illustrated in FIG. 8A: the membrane envelope comprises proteins which are anchored and exposed at its surface.

Such a virus-like particle according to the invention has the advantageous properties indicated above. In addition, when the protein or the peptide of interest b) of the fusion protein is an allergen or an allergen fragment, or more generally an antigen, the virus-like particle is efficacious in antigen presentation, and has a high capacity for activation of immune system cells. This allows effective desensitization of allergic patients. In addition, the virus-like particle according to the invention stimulates the production of allergen-specific IgGs while at the same time minimizing accessibility to basophils.

The virus-like particles according to the invention typically have a diameter of between 120 and 200 nm.

According to a third aspect, the invention relates to a method for producing a virus-like particle, comprising the expression of the fusion protein according to the invention in eukaryotic cells, preferably in plant cells.

Indeed, preferably, the method developed comprises the expression, in a plant cell, of the fusion proteins according to the invention. After their synthesis in the endoplasmic reticulum and their transportation in the endomembrane secretory system of the plant cell, these fusion proteins have the capacity to form vesicles when they are integrated into the plasma membrane. This process is identical to the budding of a virus at the surface of the cells that it infects.

One of the major advantages of this technology is that it is simple, since, after expression of the fusion proteins and extraction, the VLPs carrying the allergen of interest at their surface are preferably purified in two steps. These VLPs formed in planta have at their surface a constant density of allergens or allergen fragments. The quality of the product can thus easily be standardized and its composition is constant.

The type I or type II transmembrane fusion protein according to the invention comprises the following fragments, successively:
a) optionally, a signal peptide;
b) a protein or a peptide of interest;
c) a coiled-coil domain (or oligomerization sequence) which does not originate from a virus; and
d) a domain for anchoring in the plasma membrane, consisting of a transmembrane segment and a cytosolic segment, preferably a domain for anchoring in a plasma membrane of which at least one portion is typical of lipid rafts.

The term “fusion protein” is intended to mean a protein comprising the various fragments b) to d), and optionally a), said fragments being of different origin. In other words, fragments b) to d), and optionally a), are never present fused in the way they exist naturally.

The term “successively” is intended to mean that fragments a) to d) (or b) to d)) are present in the order a)-b)-c-d) (or b)-c)-d) or d)-c)-b)). These various fragments can be directly fused to one another, or else fused to one another via one or more linker(s). Preferably, the fusion protein according to the invention comprises a linker present between the sequences b) and c), and/or between the sequences c) and d).

The fusion protein initially contains fragments a) to d): the presence of the signal peptide enables correct trafficking of said protein into the endoplasmic reticulum. The signal peptide is then cleaved. Thus, during the budding and the formation of the VLPs according to the invention, the fusion protein no longer contains the signal peptide a), but only fragments b) to d). Consequently, the VLPs according to the invention do not contain signal peptide a). On the other hand, the description of fragments b) to d) which follows is applicable to the VLPs.

The expression “type I transmembrane protein anchored in a membrane” is intended to mean a transmembrane protein of which the N-terminal end is extracellular and the C-terminal end is cytosolic. Consequently, the type I transmembrane protein comprises, from the N-terminal to C-terminal end, optionally the signal peptide a), then the protein or the peptide of interest b), then the coiled-coil domain c) and, finally, the anchoring domain d).

The expression “type II transmembrane protein anchored in a membrane” is intended to mean a transmembrane protein of which the C-terminal end is extracellular and the N-terminal end is cytosolic. Consequently, the type II transmembrane protein comprises, from the N-terminal to C-terminal end, the anchoring domain d), then the coiled-coil domain c) and, finally, the protein or the peptide of interest b).

Preferably, the fusion protein according to the invention is a type I transmembrane protein.

### Signal Peptide a)

The signal peptide a) is any signal peptide recognized by a eukaryotic cell.

Preferably, the signal peptide is chosen from the natural signal peptide of pectate lyase and the signal peptide of tobacco chitinase.

Preferably, the signal peptide is that of tobacco chitinase, of sequence SEQ ID NO: 21.

### Protein or Peptide of Interest b)

The protein or the peptide of interest that can be used according to the invention can be any amino acid sequence which is of therapeutic or prophylactic interest.

The protein or the peptide of interest that can be used according to the invention can be any amino acid sequence which would benefit from being entirely or partially exposed at the surface of a virus-like particle, and which is capable of being recognized by immune cells, and/or of triggering a biological reaction.

The term “protein of interest” is intended to mean a sequence having at least 51 amino acids, preferably at least 100, preferably at least 200.

The term “peptide of interest” is intended to mean a sequence comprising from 2 to 50 amino acids, preferably from 5 to 45 amino acids.

The protein or the peptide of interest that can be used according to the invention is preferably chosen from:
allergens and fragments thereof. The major application of a virus-like particle containing such a protein or peptide is immunotherapy,
viral proteins and fragments thereof. The main advantage of a virus-like particle containing such a protein or peptide is vaccination,
cell surface proteins and fragments thereof. The main advantage of a virus-like particle containing such a protein or peptide may in particular be to restore an immune activity,
proteins and peptides accumulated in chronic or neurodegenerative diseases,
proteins and peptides involved in hypertension, such as angiotensinogen, angiotensin I and angiotensin II),
immunoglobulins, fragments thereof (such as Fab fragments) and derivatives thereof (such as scFv),
cytokines and fragments thereof, and
hormones and fragments thereof.

Preferably, the protein or the peptide of interest that can be used according to the invention is an allergen. Preferably, it is chosen from the allergens responsible for respiratory allergies resulting from domestic mites, such as Dermatophagoides farinae, Dermatophagoides pteronyssinus or Euroglyphus manei, allergens from storage mites such as Blomia tropicalis, allergens from mites of Acarus siro type (otherwise known as Tyroglyphus farinae), cockroach allergens, tree or grass pollen allergens, animal (cat, dog, horse) allergens, mold allergens, allergens responsible for contact allergies, such as those of hevea latex, or else allergens responsible for food allergies (milk, eggs, fish, fruit).

Among the Dermatophagoides farinae allergens, mention may be made of Der f 10, Der f 11, Der f 13, Der f 14, Der f 15, Der f 16, Der f 17, Der f 18, Der f 2, Der f 2.0101, Der f 2.0102, Der f2.0103, Der f2.0104, Der f2.0105, Der f 2.0106, Der f 2.0107, Der f 2.0108, Der f 2.0109, Der f2.0110, Der f2.0111, Der f2.0112, Der f2.0113, Derf2.0114, Derf2.0115, Der f2.0116, Der f 2.0117, Der f 20, Der f 3, Der f 4, Der f 5, Der f 6, Der f 7, Der f 8, Der f 9 and Der f HSP70.

Among the Dermatophagoides pteronyssinus allergens, mention may be made of Der p 10, Der p 11, Der p 14, Der p 15, Der p 18, Der p 2, Der p 2.0101, Der p 2.0102, Der p 2.0103, Der p 2.0104, Der p 2.0105, Der p 2.0106, Der p 2.0107, Der p 2.0108, Der p 2.0109, Der p 2.0110, Der p 2.0111, Der p 2.0112, Der p 2.0113, Der p 20, Der p 21, Der p 3, Der p 4, Der p 5, Der p 6, Der p 7, Der p 8, Der p 9.

Among the animal allergens, mention may be made of the allergens from the seminal fluid, from the epithelium, from the milk, from the saliva, from the perspiration and/or from the urine of said animals. The animals are preferably dogs, cats or horses.

Among the cat (Felis domesticus) allergens, mention may be made of Fel d 1, Fel d 1.0101, Fel d 2, Fel d 2.0101, Fel d 3, Fel d 3.0101, Fel d 4, Fel d 4.0101, Fel d 5, Fel d 5.0101, Fel d 6, Fel d 6.0101, Fel d 7, Fel d 7.0101, Fel d 8, Fel d 8.0101, Fel d Hp, Fel d IgG or Fel d S100. Among the dog (Canis familiaris) allergens, mention may be made of Can f 1, Can f 1.0101, Can f 2, Can f 2.0101, Can f 3, Can f 3.0101, Can f 4, Can f 4.0101, Can f 5, Can f 5.0101, Can f 6, Can f 6.0101, Can f 7, Can f 7.0101, Can f 8, Can f Feld1-like, Can f Homs2-like, Can f Phosvitin or Can f TCTP.

Among the horse (Equus caballus) allergens, mention may be made of Equ c 1, Equ c 1.0101, Equ c 2, Equ c 2.0101, Equ c 2.0102, Equ c 3, Equ c 3.0101, Equ c 4, Equ c 4.0101, Equ c PRVB, Equ c 10, Equ c 11, Equ c 12, Equ c 8, Equ c 9, Equ c ALA or Equ c BLG.

The sequences of these allergens are known, in particular in the Uniprot base.

Preferably, the protein or the peptide of interest that can be used according to the invention is an allergen of sequence SEQ ID NO: 22 (mature sequence of Der p 2) or SEQ ID NO: 32 (sequence of the CH1 chain of the feline allergen Fel d 1).

Preferably, the protein or the peptide of interest that can be used according to the invention is a viral protein or a fragment thereof.

Among the viral proteins, mention may in particular be made of the Zika virus envelope proteins and also the flu virus proteins, such as the hemagglutinins and the neuraminidases. Preferably, the protein or the peptide of interest that can be used according to the invention is the hemagglutinin HAI chain of sequence SEQ ID NO: 34 or the Zika virus envelope protein of sequence SEQ ID NO: 35.

Preferably, the protein or the peptide of interest that can be used according to the invention is a cell surface protein or a fragment thereof.

Among the surface proteins, mention may in particular be made of the surface antigens of tumors. Such proteins and fragments thereof are of use in particular in restoring immune activity, for example in tumor treatment.

Preferably, the protein or the peptide of interest that can be used according to the invention is a protein that is accumulated in neurodegenerative or chronic diseases.

Among the peptides that have accumulated in chronic diseases, mention may in particular be made of the ß amyloid peptide involved in Alzheimer's disease, the alpha-synuclein protein involved in Parkinson's disease, and also the CD 20, TNF-alpha or HLA (human leucocyte antigen) proteins involved in rheumatoid arthritis.

### Coiled-Coil Domain (or Oligomerization Sequence) c)

The coiled-coil domain, or oligomerization sequence, comprises several sense or antisense alpha-helix motifs which are parallel to one another and form an organized matrix that has several well-characterized biological functions. These domains are omnipresent and are found as specific domains for many types of proteins in most organisms. Coiled-coil domains from various sources can assemble to form forms which range from a dimer to a heptamer; some coiled-coil domains will adopt different polymerization levels depending on the point mutations of their amino acid sequence.

A coiled-coil domain typically consists of a repeat motif of 7 amino acids, of “hxxhcxc” type, wherein “h” is a hydrophobic amino acid, “c” is a charged amino acid, and “x” is any amino acid.

The coiled-coil domain that can be used according to the invention does not originate from a virus; it is not viral.

Among the coiled-coil domains that can be used according to the invention, mention will preferably be made of those from cortexillin, vimentin, tetrabrachion, golgins, proteins of the “Soluble N-ethylmaleimide-sensitive factor (NSF) Attachment protein REceptor” or SNARE superfamily, or else transcription factors such as GCN4 or a variant thereof, such as GCN4-pLI or GCN4-pII.

Preferably, the coiled-coil domain is that from the GCN4, GCN4-pLI or GCN4-pII transcription factor.

Preferably, the coiled-coil domain is chosen from SEQ ID NO: 24 (GCNA-pII trimerization sequence of yeast GCN4 transcription factor), SEQ ID NO: 27 (GCN4-pLI tetramerization sequence of yeast GCN4 transcription factor), SEQ ID NO: 28 (GCN4-pAA heptamerization sequence of yeast GCN4 transcription factor), SEQ ID NO: 29 (IZN4 glycosylated oligomerization sequence of yeast GCN4 transcription factor), SEQ ID NO: 33 (synthetic sequence mimicking a coiled-coil) and SEQ ID NO: 30 (SNARE oligomerization sequence).

### Domain for Anchoring in the Plasma Membrane (or Transmembrane Domain) d)

The transmembrane domain is a short sequence of lipophilic amino acids which interacts with the specific lipids of the plasma membrane components. Preferably, the plasma membrane comprises at least one portion typical of lipid rafts.

These anchoring domains are common (but not through a consensus sequence) to the surface proteins of viruses, but also to proteins which are naturally integrated into the membrane of the living cells. Each transmembrane domain participates in the bending and the budding of the plasma membrane.

Among the anchoring domains that can be used according to the invention, mention will preferably be made of those from the proteins listed in table 1A:

TABLE 1A
Transmembrane proteins

| | | Number of TM |
|---|---|---|
| Transmembrane (TM) proteins | Examples (Uniprot references) | domains |
| Leucine-rich repeat receptor-like protein | Q9M7A8 | 1 |
| kinase NtTMK1 | | |
| Caveolin | Q03135 | 1 |
| BRI1-associated receptor kinase 1 (BAK1) | Q94F62 | 1 |
| Receptor kinases | Q9LDG0, Q9ZT08, Q8LD58, Q7XHW7, | 1 |
| | Q8H811, Q9SUQ3, Q5ZBN0 | |
| Calcium-dependent protein kinases | Q6KC54, Q6EE26, Q5EDD1, P28582, | 1 |
| | Q9ARI5, Q7XZK4, Q8GSB1, Q9FWF0, | |
| | Q94KH6 | |
| NtRac2/NTGP3 | Q9ZRD2 | 1 |
| ARF1-like GTP-binding protein | Q9M7P4 | 1 |
| Stomatin | Q93VP6, Q60634 | 1 |
| Ascorbate peroxidase | Q8W4V7 | 1 |
| LAT | O43561 | 1 |
| VIP36 | P49256 | 1 |
| Elicitor-inducible protein (EIG-J7) | Q9FXT1 | 1 |
| Hsp90-2 Chaperone | Q6UJX5 | 1 |
| Syntaxin | Q9SF29, Q9SRV7, Q9ZSD5 | 1 |
| Phragmoplastin | Q9SMB6 | 1 |
| Fasciclin-like arabinogalactan protein 8 | O22126 | 1 |
| (precursor) | | |
| Ras-related protein RAB8-3 | Q8W3J3 | 2 |
| Ras-related protein RAB8-5 | Q8W3J2 | 2 |
| Flotillin | Q501E6, 075955 | 2 |
| Harpin-inducing protein 1 | Q6L7J8, Q6L7J7 | 2 |
| Pectinesterase-like | Q9FHN6 | 2 |
| Protein kinase | Q9SH35 | 3 |
| Endo-1,4-β-glucanase | O04890 | 3 |
| MAL | P21145 | 4 |
| Synatophysin | Q62277 | 4 |
| Prominin | O43490 | 5 |
| Aquaporin | O09224, O09222, Q40595, Q8W506, | 6 |
| | O24662, Q9FPZ6, Q9FPZ7 | |
| NADPH oxidase NtrbohD | Q8RVJ9 | 6 |
| Respiratory burst oxidase homolog StrbohB | Q948T9 | 6 |
| Lysine- and histidine-specific transporter | O24405 | 8 |
| LHT1 | | |
| Glucan synthase | Q9SJM0, Q8S8D4, Q5VS25, Q9ZT82, | 8 |
| | Q9SFU6 | |
| Callose synthase | Q8H046, Q9LXT9, Q9LTG5, Q9XEG1 | 9 |
| Plasma membrane ATPase | Q42932, Q08436, Q03194, Q5U9D4, | 10 |
| | Q9SWH2, Q9SWH0 | |
| Calcium-transporting ATPase | Q9LU41 | 10 |
| Ammonium transporter | P58905 | 11 |
| MDR-like ABC transporter mdr8 | Q7FMW3 | 12 |
| P-glycoprotein-like protein pgp3 | Q9SY12 | 12 |
| ABC transporter | O80725, Q9FWX8 | 12 |
| Phosphate transporter | Q9ST22, Q9LLS5, Q9AYT1 | 12 |
| Transmembrane protein PT3 | Q8W4W9 | 12 |
| H+/monosaccharide co-transporter MST1 | Q06312 | 12 |
| High affinity nitrate transporter protein | Q84MZ8 | 12 |
| PDR-type ABC transporter 1 NtPDR1 | Q76CU2 | 12 |

Among the anchoring domains that can be used according to the invention, mention will preferably be made of those from the viral envelope proteins listed in table 1B below:

| Family | Examples | Envelope proteins |
|---|---|---|
| Flavivirus | Dengue virus | Protein E |
| | Yellow fever virus | |
| | Saint Louis encephalitis virus | |
| | Japanese encephalitis virus | |
| | West Nile virus | |
| | Zika virus | |
| | BYD virus (identified in China in 2010, which affects | |
| | ducks) | |
| Togavirus | Sindbis virus | GP |
| | Eastern equine encephalitis virus | |
| | Western equine encephalitis virus | |
| | Ross River virus | |
| | O'nyong'nyong virus | |
| Retrovirus | Oncovirus (5 genera) | GP41/120 |
| | Lentivirus (such as HIV) | |
| | Spuma virus | |
| Coronavirus | Canine Corona virus | S protein and HE |
| | Feline Corona virus | (hemagglutinin-esterase) |
| | Transmissible gastroenteritis virus in pigs | protein |
| | Porcine respiratory virus | |
| | Bowing Corona virus | |
| | Human Corona virus | |
| | Murine hepatitis virus | |
| | Rat sialodacryoadenitis virus | |
| Filovirus | Ebola virus | Glycoprotein |
| | Marburg virus | |
| Rhabdovirus | Rabies virus | GP |
| | Viral hemorrhagic virus | |
| | VSV-EBOV | |
| | Beet disease | |
| Bunyavirus | Hantaan virus | Gn/Gc |
| | Dugbe virus | |
| | Rift valley fever | |
| | Tomato spotted wilt virus | |
| Orthomyxovirus | Influenza virus (myxoinfluenza) | Hemagglutinin/ |
| | | neuraminidase |
| Paramyxovirus | Mumps virus | Fusion protein F |
| | Sendai virus | Attachment protein |
| | SV5 virus | (HN, H or G) |
| | Newcastle disease virus | |
| | Measles virus | |
| | Distemper virus | |
| | Rinderpest virus | |
| | Respiratory syncytial virus (VRS) | |
| | Bovine respiratory syncytial virus (VRSB) | |
| | Parainfluenza | |
| Arenavirus | Lassa fever | GP |
| | Argentine hemorrhagic fever | |
| | Bolivian hemorrhagic fever | |
| | Brazilian hemorrhagic fever | |
| | Venezuelan hemorrhagic fever | |
| Hepadnavirus | Hepatitis B virus (HBV) | GPL, S or M |
| Herpesvirus | Herpes simplex virus (HSv) | Glycoprotein |
| | EHV-1 (equine herpes virus) | gD, gB, gH, gL, gC |
| | Genital herpes virus | |
| Poxvirus | Orthopoxvirus; species type: vaccinia virus; disease: | GP41/120 |
| | cowpox, pox, smallpox | |
| | Parapoxvirus; species type: Orf virus | |
| | Avipoxvirus; species type: Fowlpox virus | |
| | Capripoxvirus; species type: Sheeppox virus | |
| | Leporipoxvirus; species type: Myxoma virus | |
| | Suipoxvirus; species type: Swinepox virus | |
| | Molluscipoxvirus; species type: Molluscum contagiosum | |
| | virus | |
| | Yatapoxvirus; species type: Yaba monkey tumor virus | |

Preferably, the anchoring domain that can be used according to the invention is chosen from the anchoring sequence of the H5N1 influenza virus H5 hemagglutinin (SEQ ID NO: 26) and the anchoring sequence of the PDLP1 protein (A0A0D3D8S3) (SEQ ID NO: 31).

### Linkers

Preferably, the fusion protein according to the invention comprises a linker present between fragments b) and c), and/or between fragments c) and d).

Linkers are short sequences of amino acids (2 to 10 amino acids, preferably 2 to 6) which create a flexible arm. They may be useful for creating a flexible space between the anchoring domain and the spiraling of the coiled-coil domain, if the fact that the two domains are too close together interferes with correct assembling. They are not required under conditions where a direct link between the two domains (anchoring and coiled-coil domains) does not interfere with the overall three-dimensional structure of the fusion protein.

Preferably, the linker is a sequence of -(GGGS)ₙ-type, wherein n is an integer. Preferably, the linker is chosen from SEQ ID NO: 23 (n=2) and SEQ ID NO: 25 (n=1).

Thus, preferably, the fusion protein according to the invention is such that:
a) the optional signal peptide has the sequence SEQ ID NO: 21;
b) the protein or the peptide of interest is chosen from:
allergens and fragments thereof,
viral proteins and fragments thereof,
cell surface proteins and fragments thereof,
proteins and peptides that are accumulated in chronic or neurodegenerative diseases,
proteins and peptides involved in hypertension,
immunoglobulins and fragments thereof,
cytokines and fragments thereof, and
hormones and fragments thereof;
c) the coiled-coil domain is chosen from SEQ ID NO: 24, SEQ ID NO: 27, SEQ ID NO: 28, SEQ ID NO: 29, SEQ ID NO: 33 and SEQ ID NO: 30; and
d) the anchoring domain is chosen from the anchoring sequence of the H5N1 influenza virus H5 hemagglutinin (SEQ ID NO: 26) and the anchoring sequence of the PDLP1 protein (SEQ ID NO: 31).

Thus, preferably, the fusion protein according to the invention comprises, preferably consists of, a sequence chosen from SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18 and SEQ ID NO: 20.

Likewise, preferably, the VLP according to the invention comprises:
an envelope consisting of a plasma membrane of which at least one portion has a composition typical of lipid rafts; and
at least one type I or II transmembrane fusion protein anchored in said membrane, said fusion protein comprising the following fragments, successively:
b) the protein or the peptide of interest chosen from:
allergens and fragments thereof,
viral proteins and fragments thereof,
cell surface proteins and fragments thereof,
proteins and peptides that are accumulated in chronic or neurodegenerative diseases,
proteins and peptides involved in hypertension,
immunoglobulins and fragments thereof,
cytokines and fragments thereof, and
hormones and fragments thereof;
c) the coiled-coil domain chosen from SEQ ID NO: 24, SEQ ID NO: 27, SEQ ID NO: 28, SEQ ID NO: 29, SEQ ID NO: 33 and SEQ ID NO: 30; and
d) the anchoring domain chosen from the anchoring sequence of the H5N1 influenza virus H5 hemagglutinin (SEQ ID NO: 26) and the anchoring sequence of the PDLPI protein (SEQ ID NO: 31):
fragments b) and c) being exposed on the outside of the VLP.

A subject of the present invention is also the nucleic acids (or a nucleotide sequence) encoding the fusion protein.

Once the nucleotide sequence has been obtained, the latter is placed in an expression vector using conventional methods. A subject of the present invention is thus also a vector comprising the nucleic acid encoding the fusion protein. The selection of a suitable expression vector will depend on the method for introducing the expression vector into host cells. A typical expression vector contains eukaryotic DNA elements, such as a transcription initiation sequence for the exogenous gene, for instance a promoter, and DNA elements which control the processing of the transcripts, such as termination/polyadenylation sequences, and an expression cassette allowing for the expression of a silencing inhibitor. It also contains sequences such as t-DNAs which are required for the integration of a piece of DNA into the plant or into the plant cell.

Preferably, the expression vector comprises:
at least one nucleotide sequence encoding the fusion protein, preferably functionally linked to a strong promoter, preferably a 35S promoter;
an expression cassette allowing the expression of a silencing inhibitor, preferably p19; and
DNA elements which control the processing of the transcript, such as termination/polyadenylation sequences, preferably the Tnos sequence (nopaline synthase termination sequence).

The expression vector is preferably pAG01.

The promoters used for controlling the expression of the fusion protein are strong promoters, and may be plant gene promoters, such as for example the ubiquitin promoter, the ribulose-1, 5-bisphosphate carboxylase small subunit promoter, Agrobacterium tumefaciens promoters, the nopaline synthase and octopine synthase promoters, or else viral promoters such as cauliflower mosaic virus (CaMV) 19S and 35S. Preferably, the strong promoter is 35S.

A subject of the present invention is also a host cell comprising at least one nucleic acid encoding the fusion protein. The host cell may be a plant cell.

A subject of the present invention is also a method for producing virus-like particles (VLPs) comprising the expression of the nucleic acid encoding the fusion protein in eukaryotic cells, preferably plant cells.

The general methods for culturing plants, and also the methods for introducing expression vectors into a plant tissue, are available to those skilled in the art. They are varied and depend on the plant selected. Preferably, the plants will be cultivated according to the techniques specific for the Allergopur platform. This method for producing recombinant proteins is described in application FR 1 255 510, and comprises a first step of culturing the plant, under aeroponic or hydroponic conditions and under LED lighting. After this first step, the agroinfiltration of the plants is carried out under vacuum, using agrobacteria comprising a DNA fragment encoding the fusion protein according to the invention. This agroinfiltration step can be carried out by any means for producing a vacuum. Preferably, in the method used according to the invention, it is carried out under vacuum by Venturi effect. Among the agrobacteria that can be used according to the invention, mention is preferably made of the LBA4404, GV3101, EHA 101/105 or C58 strains.

Once the agroinfiltration step has been carried out, the plants are put back in culture, typically for 3 to 6 days, ideally while providing frequent misting of said plants for the first 6 hours of culture following the agroinfiltration. The VLPs are then extracted and purified as described below.

The VLP extraction can be carried out by enzymatic extraction. This method is an adaptation of the method described in particular in application WO 2014/153674. Preferably, the enzymatic extraction of the VLPs is carried out by means of the following steps:
infiltration under vacuum (in particular as described above for the agroinfiltration) of the aerial part of plants (i.e. the leaves), in an enzymatic solution containing pectocellulosic enzymes, which does not exhibit any proteolytic activity; preferably, a mixture of pectinases and cellulases which is formulated at 4% in a medium comprising 50 mM of sodium citrate, pH 5.2, 0.5 M NaCl and 0.04% metabisulfite. Preferably, the macerozyme is formulated at 0.5% in a medium comprising 50 mM of sodium citrate, pH 5.2, 0.5 M NaCl and 0.04% metabisulfite,
the leaves are subsequently sampled and then incubated in the enzymatic solution,
the mixture is placed with shaking on an orbital shaker between 20 and 30 rpm at ambient temperature (i.e. approximately 20-23° C.) for a period of between 30 minutes and 2 h,
the digestate is then filtered, preferably on a 2-3 mm then 250 μm cloth, then optionally continuously centrifuged (for example at 1000×g for 2-5 minutes), and the supernatant is recovered in order to perform a tangential filtration.

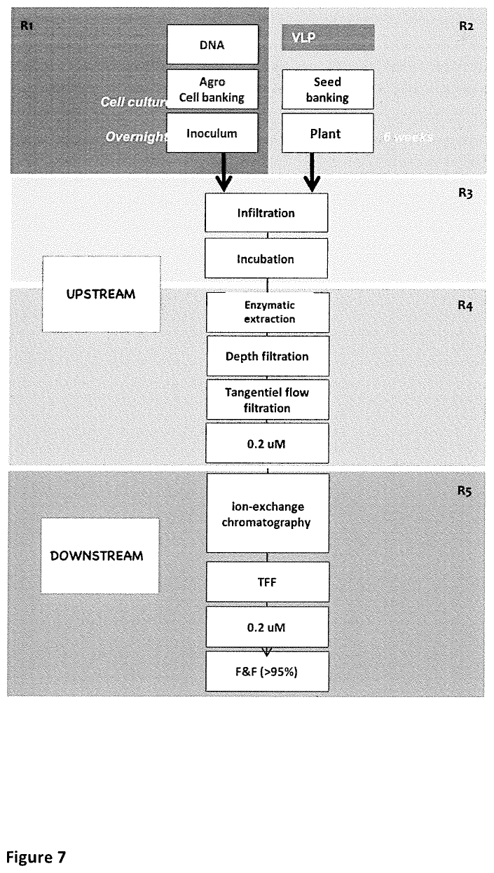

Such a method is illustrated in FIG. 7.

Thus, preferably, a subject of the invention is also a method for producing virus-like particles (VLPs) according to the invention in a plant cell or a plant, comprising the following steps:
a) transformation of agrobacteria with an expression vector comprising a nucleotide sequence encoding a fusion protein according to the invention functionally linked to a strong promoter; and
b) transfection of the plant cell or the plant with the agrobacteria obtained in step a).

The transformation of step a) is typically carried out using methods known to those skilled in the art, for example by means of heat shocks with successive passages at 4° C., −80°° C. and 37° C.

The transfection of step b) preferably comprises the following steps:
b1) culture of the plant cell or the plant, under aeroponic or hydroponic conditions, and under LED lighting, preferably for four to six weeks,
b2) agroinfiltration of the plant cell or plant obtained in b1) under vacuum, with the agrobacteria obtained in step a). This agroinfiltration step is preferably carried out under vacuum by Venturi effect,
b3) return to culture of the plant cell or the plant obtained in b2), typically for 3 to 6 days, in order to obtain the virus-like particles.

Finally, the VLPs obtained are extracted and purified, in particular by enzymatic extraction as described above.

A subject of the present invention is also a virus-like particle comprising:
an envelope consisting of a plasma membrane of which at least one portion has a composition typical of lipid rafts; and
at least one fusion protein according to the invention without signal peptide a), anchored in said membrane.

Such a virus-like particle (VLP) thus comprises:
an envelope consisting of a plasma membrane of which at least one portion is typical of lipid rafts; and
at least one type I or II transmembrane fusion protein anchored in said membrane, said fusion protein comprising the following fragments, successively:
b) a protein or a peptide of interest;
c) a coiled-coil domain (or oligomerization sequence) which does not originate from a virus; and
d) a domain for anchoring in the plasma membrane, consisting of a transmembrane segment and a cytosolic segment, preferably a domain for anchoring in a plasma membrane of which at least one portion is typical of lipid rafts,
fragments b) and c) being exposed on the outside of the VLP.

The expression “portion of plasma membrane typical of lipid rafts” is intended to mean a phospholipid bilayer (i.e. plasma membrane) found in the microdomains of lipid rafts. Such a bilayer is rich in cholesterol and in phospholipids, preferably in phosphatidylcholine and in phosphatidylethanolamine, and in sphingolipids, such as sphingomyelin, but poor in docosahexaenoic acid. In addition, it has a low density, and is insoluble in mild detergents (for example polysorbates).

The unique characteristics of such a VLP are an undetectable immunogenicity (other than the protein or the peptide of interest b), its ability to spontaneously self assemble, its specific content of lipids, preferably typical of lipid rafts, the fact that its membrane is very poor in host-cell membrane proteins, its ability to be expressed at high yields in numerous eukaryotic cell types (leaves, insects or plants) and its ease of preparation.

The VLP according to the invention may be used in therapy. It may be used as a medicament. It may also be used in allergen immunotherapy (AIT).

The sequences listed in the present application are summarized in the table below:

| SEQ ID NO: | Definition |
|---|---|
| 1 | cDNA encoding the natural form of Der p2 |
| 2 | Der p2 protein |
| 3 to 20 | cDNA and fusion proteins according |
| | to the invention |
| 21 | Tobacco chitinase signal peptide |
| 22 | Mature sequence of Der p2 |
| 23 | Linker |
| 24 | GCN4-pII coiled-coil domain |
| 25 | Linker |
| 26 | Anchoring sequence of the H5N1 influenza |
| | virus H5 hemagglutinin |
| 27 | GCN4-pLI coiled-coil domain |
| 28 | GCN4-pAA coiled-coil domain |
| 29 | IZN4 coiled-coil domain |
| 30 | SNARE coiled-coil domain |
| 31 | Anchoring sequence of the PDLP1 protein |
| 32 | Sequence of the CH1 chain of the feline |
| | allergen Fel d1 |
| 33 | Synthetic coiled-coil domain |
| 34 | Hemagglutinin HA1 chain |
| 35 | Zika virus envelope protein |

The examples which follow illustrate, but are not intended to limit, the scope of the invention. It would be obvious for those skilled in the art that variants and modifications are possible and fall within the context and spirit of the invention.

The figure legends are the following:

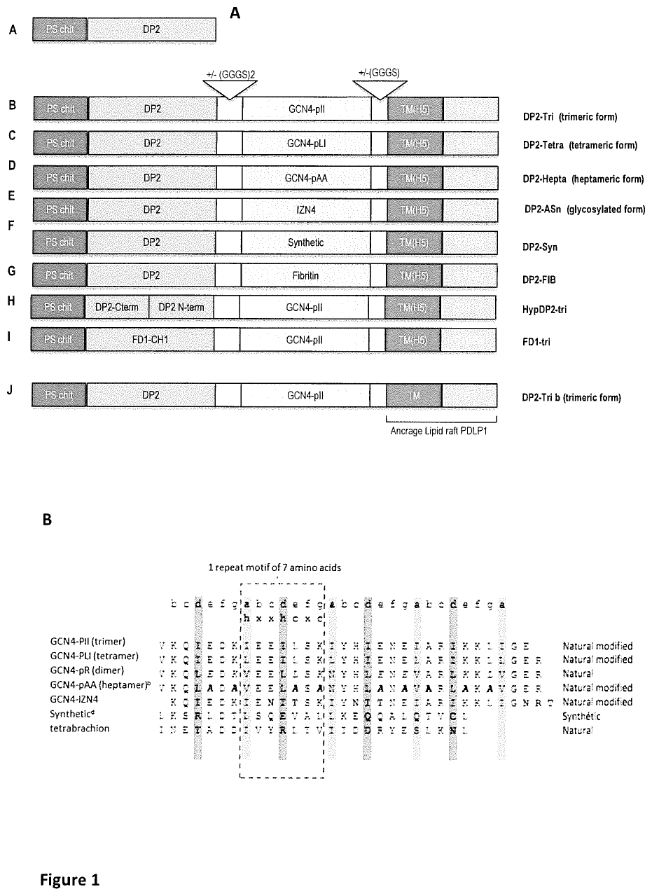

FIG. 1: Diagrammatic representation of the various expression cassettes for producing an allergen linked to an oligomerization sequence and to a sequence for anchoring the plasma membrane, preferably at the level of the lipid rafts
A) The cDNA encoding the optimized, preferably harmonized, Der p2 (DP2, SEQ ID NO: 22) is linked to 1) the cDNA encoding the tobacco chitinase signal peptide (PS Chit, SEQ ID NO: 21), 2) an oligomerization (coiled-coil) sequence from a transcription factor (GCN4-pII/trimeric form, B-GCN4-PLI/tetrameric form, C-GCN4-IZN4/glycosylated form,
E-GCN4-pAA/heptameric form, D) or from any other family of proteins having a coiled-coil sequence (SNARE, Golgin, Fibritin, G) or a synthetic sequence mimicking a coiled-coil sequence (F), and finally 3) an anchoring sequence from enveloped virus envelope protein (TM/CT of influenza H5, B to I) or from type I protein anchored in the lipid rafts (“lipid raft”) (J).
B) Diagram representing the structure of the oligomerization or coiled-coil sequence. This sequence consists of a repeat motif of 7 amino acids, of “hxxhcxc” type, wherein “h” is a hydrophobic amino acid, “c” is a charged amino acid, and “x” is any amino acid.

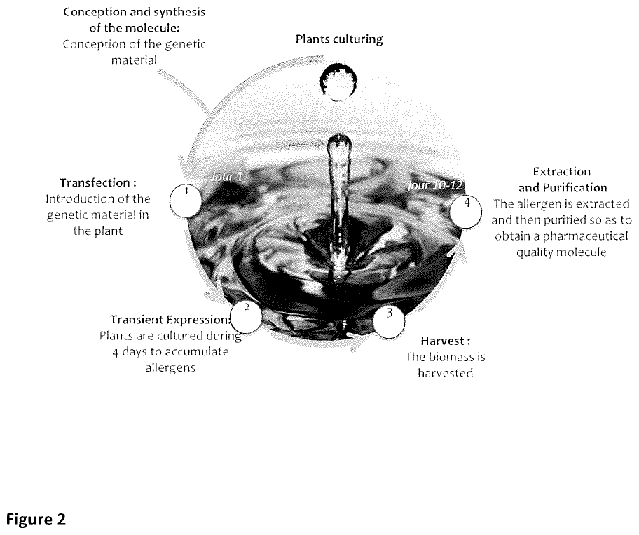

FIG. 2: AllergoPur platform used for the expression and production of the various forms of VLP

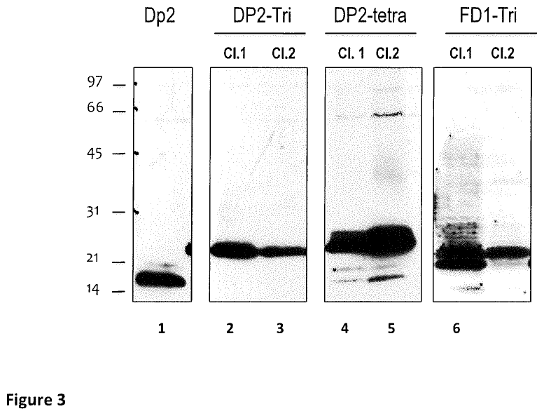

FIG. 3: Production of the proteins described in FIG. 1A
The proteins extracted from plants transfected under vacuum for the expression of the DP2(lane 1), DP2-Tri (lanes 2-3) DP2-Tetra (lanes 4-5) or FD1-Tri (lanes 6-7) proteins were analyzed by immunodetection with an antibody directed against the Der p2 or Fel d1 allergen. The immunodetection analysis demonstrates the specific production of the proteins, the molecular weight of which corresponds to the expected weight. Two clones of agrobacteria (Cl.1 and Cl.2) were analyzed for each construct.

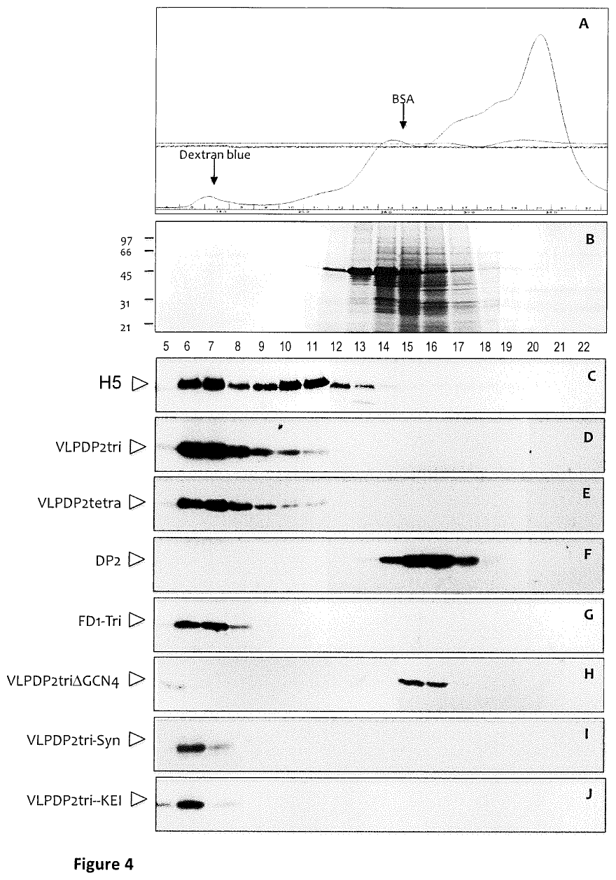

FIG. 4: Purification and characterization of the VLPs carrying the allergens by size exclusion chromatography

Protein extracts of leaves producing DP2-Tri (panel D), DP2-Tetra (panel E), DP2 soluble (panel F) FD1-Tri (panel G), DP2triDGCN4 (GCN4 deletion, panel H), DP2tri-Syn (GCN4 replacement, panel I) and DP2tri-KEI (GCN4 replacement, panel J) were separated by chromatography on a calibrated S-500/HR column. The total soluble protein content of each fraction was evaluated by spectrometry (panel A) and staining with Coomassie blue after separation by SDS-PAGE (panel B). The allergen content of the elution fractions was revealed by immunological detection using anti-Der p2 or anti-Fel d1 antibodies. Protein extracts of leaves producing hemagglutinin in the form of VLPs from H5N1 (panel C) were separated by gel filtration on a calibrated S-500/HR column and are used as controls.

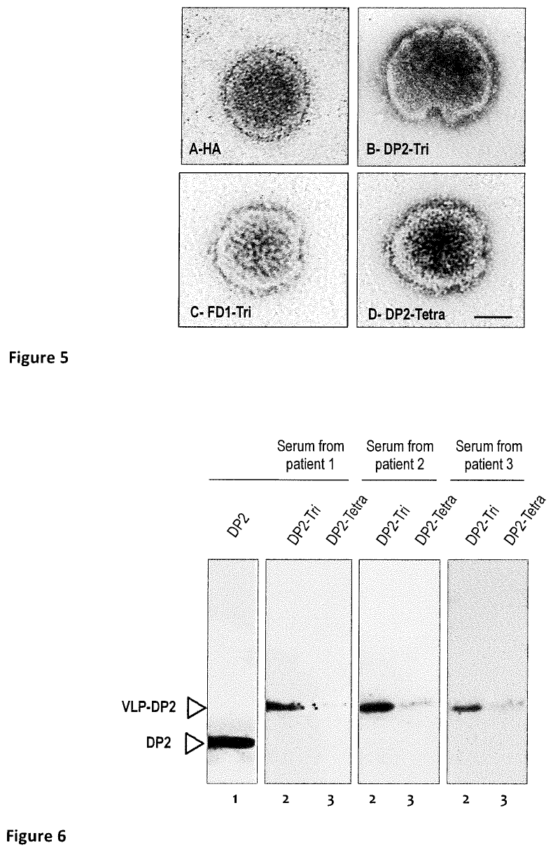

FIG. 5: Characterization of the VLPs carrying the isolated allergens by means of examination by electron microscopy and negative staining
The VLPs carrying the allergens have a morphology and a size that are very close to those described for influenza virions.
The bar represents 50 nm.

FIG. 6: Reactivity of the allergens produced in the form of VLPs with sera from patients allergic to Der p2
The proteins extracted from plants transfected under vacuum for the expression of the DP2 (lane 1), DP2-Tri (lane 2) and DP2-Tetra (lane 3) proteins were analyzed by immunodetection with sera from patients allergic to Der p2. The immunodetection analysis demonstrates the recognition of the allergens carried by the VLPs by the IgEs of the patient sera.

FIG. 7: Method for large-scale VLP production

FIG. 8: Structure of the VLPs and of the fusion proteins assembled according to the invention
A) Structure of a VLP according to the invention. The VLP consists of a plasma membrane envelope, in which the fusion proteins according to the invention are attached. The protein or the peptide of interest (for example the allergen) is thus exposed at its surface.
B) Structure of the fusion proteins according to the invention, assembled within the VLP. The oligomerization sequences allow the fusion proteins to form polymers (for example in this case the allergen, A) at the surface of the VLP.

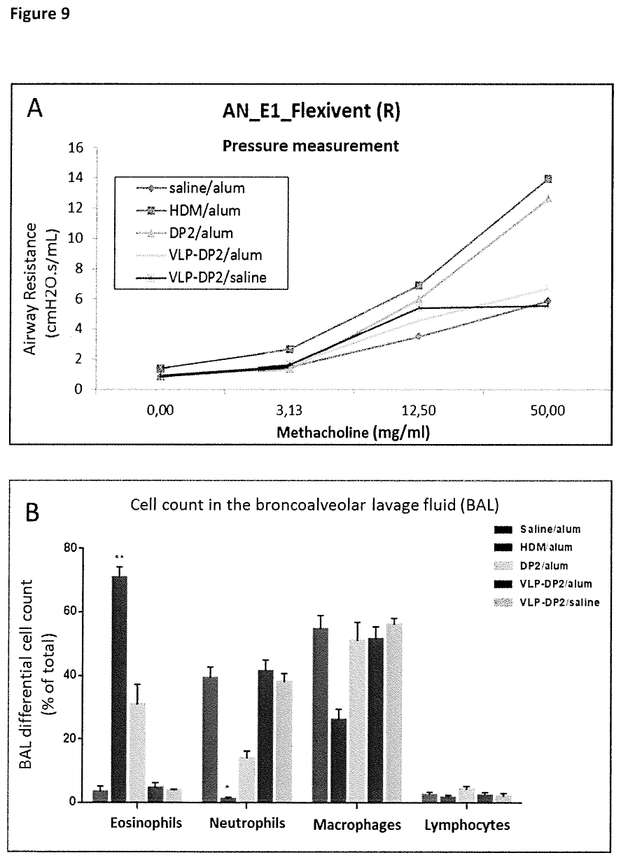

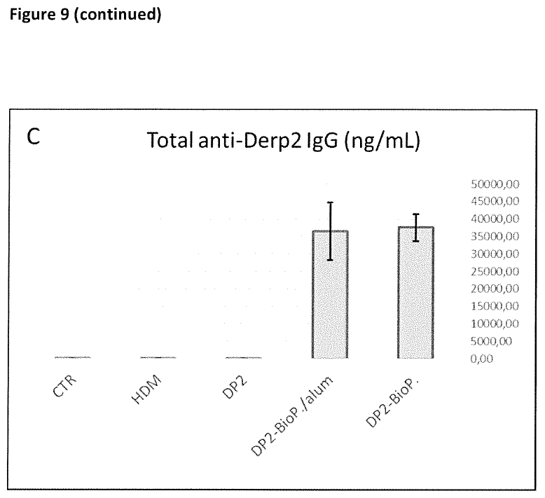

FIG. 9: The antigens conjugated to VLP have a very strong immunogenic power but no allergenicity
Panel A: Evaluation of the hyperactivity of the airways induced by the Der p2 allergen, by the Flexivent method. The mice (n=10/group) was sensitized with the Der p2 allergen in soluble form (DP2-Alum) or in VLP form (DP2-VLP/alum and DP2-VLP/saline) and challenged with a mite extractor. Twenty-four hours after the final challenge, the airway hyperactivity to inhaled methacholine was determined by the Flexivent method. The pulmonary reactivity triggered in the presence of the allergen in VLP form is comparable to the control mice.
Panel B: Counting of the inflammatory cells in the respiratory pathways, collected by bronchoalveolar lavage (BAL) of the lung. The mice (n=10/group) were sensitized with the Der p2 allergen in soluble form (DP2-Alum) or in VLP form (DP2-VLP/alum and DP2-VLP/saline) and challenged with a mite extract. Twenty-four hours after the final challenge,
the BAL cells were collected and the cells were counted (eosinophils; neutrophils; macrophages; lymphocytes). The neutrophils are very predominant in the mice having received the Der p2 allergen in soluble form.
Panel C: Assaying of the Der p2-specific IgGs. The mice (n=10/group) were sensitized with the Der p2 allergen in soluble form (DP2-Alum) or in VLP form (DP2-VLP/alum and DP2-VLP/saline) and challenged with a mite extract. Twenty-four hours after the final challenge, the IgGs were measured in the BAL fluid and the blood serum that was collected by cardiac puncture. The mice having received injections of DP2-VLP with or without adjuvant have an IgG titer which is one thousand times higher than the mice having received the soluble Der p2.

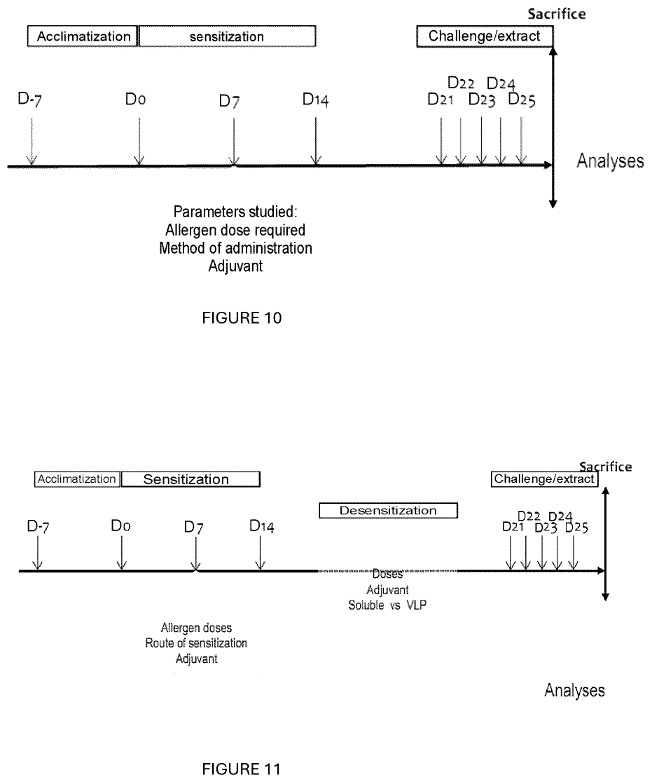

FIG. 10: Protocol used for Example 9

FIG. 11: Protocol used for Example 10

### EXAMPLES

### Example 1: Molecular Design and Synthesis of the Genes

The cDNAs are synthesized by optimizing and then harmonizing the codon usage for their recognition by the plant system. In the context of this invention, the preferred optimization is the optimization for expression in Nicotiana benthamiana.

The constructs are illustrated in FIG. 1. In particular:
A: cDNA encoding the natural form of the protein (SEQ ID NO: 1). This cDNA may or may not be fused to trafficking signals described in patent WO 2008/056265. The corresponding protein has the sequence SEQ ID NO: 2.
B: cDNA encoding the mature form of the Der p2 allergen (SEQ ID NO: 22) fused to the tobacco chitinase signal sequence (Neuhaus, J.-M. 1996), to the GCN4-pII trimerization signal of the yeast GCN4 transcription factor and to the anchoring sequence of the H5N1 influenza virus H5 hemagglutinin (SEQ ID NO: 3). The corresponding protein has the sequence SEQ ID NO: 4.
C: cDNA encoding the mature form of the Der p2 allergen (SEQ ID NO: 22) fused to the tobacco chitinase signal sequence (Neuhaus, J.-M. 1996), to the GCN4-pLI tetramerization sequence of the yeast GCN4 transcription factor and to the anchoring sequence of the H5N1 influenza virus H5 hemagglutinin (SEQ ID NO: 5). The corresponding protein has the sequence SEQ ID NO: 6.
D: cDNA encoding the mature form of the Der p2 allergen (SEQ ID NO: 22) fused to the tobacco chitinase signal sequence (Neuhaus, J.-M. 1996), to the GCN4-pAA heptamerization sequence of the yeast GCN4 transcription factor and to the anchoring sequence of the H5N1 influenza virus H5 hemagglutinin (SEQ ID NO: 7). The corresponding protein has the sequence SEQ ID NO: 8.
E: cDNA encoding the mature form of the Der p2 allergen (SEQ ID NO: 22) fused to the tobacco chitinase signal sequence (Neuhaus, J.-M. 1996), to the IZN4 glycosylated oligomerization sequence of the yeast GCN4 transcription factor and to the anchoring sequence of the H5N1 influenza virus H5 hemagglutinin (SEQ ID NO: 9). The corresponding protein has the sequence SEQ ID NO: 10.
F: cDNA encoding the mature form of the Der p2 allergen (SEQ ID NO: 22) fused to the tobacco chitinase signal sequence (Neuhaus, J.-M. 1996), to a synthetic sequence mimicking a coiled-coil and to the anchoring sequence of the H5N1 influenza virus H5 hemagglutinin (SEQ ID NO: 11). The corresponding protein has the sequence SEQ ID NO: 12.
G: cDNA encoding the mature form of the Der p2 allergen (SEQ ID NO: 22) fused to the tobacco chitinase signal sequence (Neuhaus, J.-M. 1996), to a SNARE oligomerization sequence and to the anchoring sequence of the H5N1 influenza virus H5 hemagglutinin (SEQ ID NO: 13). The corresponding protein has the sequence SEQ ID NO: 14.
H: cDNA encoding two fragments of Der p2 (SEQ ID NO: 22) that are fused to the tobacco chitinase signal sequence (Neuhaus, J.-M. 1996), to the GCN4-pII trimerization sequence of the yeast GCN4 transcription factor and to the anchoring sequence of the H5N1 influenza virus H5 hemagglutinin (SEQ ID NO: 15). The corresponding protein has the sequence SEQ ID NO: 16.
I: cDNA encoding the CHI chain of the Fel dl allergen (SEQ ID NO: 32) fused to the tobacco chitinase signal sequence (Neuhaus, J.-M. 1996), to the GCN4-pII trimerization sequence of the yeast GCN4 transcription factor and to the anchoring sequence of the H5N1 influenza virus H5 hemagglutinin (SEQ ID NO: 17). The corresponding protein has the sequence SEQ ID NO: 18.
J: cDNA encoding the mature form of the Der p2 allergen (SEQ ID NO: 22) fused to the tobacco chitinase signal sequence (Neuhaus, J.-M. 1996), to the GCN4-pII trimerization sequence of the yeast GCN4 transcription factor and to the anchoring sequence of the PDLPI protein (A0A0D3D8S3) of lipid rafts (SEQ ID NO: 19). The corresponding protein has the sequence SEQ ID NO: 20.

### Example 2: Plasma Preparation

Xba I/kpn I and Sal I/Sac I restriction sites are respectively integrated at the 5′ and 3′ ends of the cDNA during the synthesis. These sites are then used in order to clone the cDNAs into the pAG01 binary expression vector. The cDNAs are cloned upstream of a 35S promoter (35S) and downstream of a nopaline synthase termination sequence (tnos); the pAGO1 vector also contains an expression cassette which makes it possible to express the p19 silencing inhibitor simultaneously with the recombinant protein in order to increase the production yields. The vectors are then used to transform the LBA4404 strain of Agrobacterium tumefaciens.

### Example 3: Transient Expression of Der p2 Produced in VLP Form in Nicotiana benthamiana Leaves—Use of the AllergoPur Platform

For the production by transient expression, Agrobacterium tumefaciens LBA4404 is used for the transfer of a cDNA encoding Der p2 linked to an oligomerization sequence and to an anchoring sequence without the gene of interest being integrated into the genome of the plant cell. This is referred to as transfection and not transgenesis. The plants are cultured under hydroponic conditions in the presence of a nutritive medium (GHE, floragrow, floramicro, florabloom, 10 ml/15 ml/5 ml per 10 1 of osmosed water) and under LED lighting.

The agrobacterium is transferred into the foliar tissue via agroinfiltration according to two methods. For the production of small batches of proteases intended for prototype screening, the agrobacteria are manually injected by means of a syringe applied against the epidermis of the lower face of the leaf. Foliar disks sampled from the leaves 4 to 6 days after the agroinfiltration are used for the analysis of the various VLP prototypes. This screening step makes it possible to define the expression vector that will be used for obtaining the Der p2 allergen anchored in the membrane of optimal quality. The same method is used for large-scale production, but in this case, the agroinfiltration is carried out under vacuum, in chambers containing several liters of a culture of agrobacteria and wherein several tens of plants are simultaneously infiltrated. These plants are then put back into culture for 3-6 days before purification of the VLPs carrying the allergen (FIG. 2).

### Example 4: Production of the VLPs Carrying the Der p2 Allergen

The expression of the proteins produced in example 3 and also the yields are respectively analyzed by Western blotting and ELISA. The results are given for 3 allergens produced in VLP form (DP2-tri; DP2-tetra and FD1-tri) (FIG. 3).

### Example 5: Evaluation of the VLP Formation/Size Distribution Analysis

A size distribution analysis of the structures carrying the Der p2 (DP2-tri/DP2-tetra) or Fel d1 (FD1-Tri) allergens was carried out. After infiltration under vacuum of N. benthamiana plants with the Agrobacterium strain LBA4404, as described in example 3, the total protein extracts were separated by size exclusion chromatography on an S-500 (HR) high-resolution column (GE Healthcare Bio-Science Corporation). The elution fractions were controlled with respect to their total protein content and to their VLP-allergen content by Western blotting with anti-allergen antibodies. For all the extracts analyzed, the soluble protein concentration in the eluate reaches a maximum in fractions 14-16 (FIG. 4). On the other hand, the Western blotting analysis demonstrates an accumulation of allergens in fractions 6 and 7 (that is to say before the elution of the Dextran Blue used as marker) which shows the linking of the allergens to very high molecular weight structures in the 2 MDa zone.

The 32 ml Sephacryl S-500/HR columns (GE Healthcare Bio-Science Corporation) were equilibrated with 50 mM PBS, pH 7.4, 150 mM NaCl. Samples of total protein extracts of 1.5 ml were loaded and then eluted with the equilibration buffer. Twenty-four 1.5 ml elution fractions were collected and analyzed for the content in proteins measured by absorbance spectrophotometry at 280 nm. The proteins of each fraction were concentrated by precipitation with acetone and then redissolved in one and the same volume of elution buffer before the analysis by SDS-PAGE and Western blotting. The elution profiles of the Dextran Blue 2000 and of the soluble proteins were compared for each chromatogram in order to be sure of the reproducibility of this separation technique.

### Example 6: VLP Morphology-Analysis by Electron Microscopy

The transmission electron microscopy of the purified product (resulting from the production in example 3 followed by purification) indicates that the high-molecular-weight structures isolated by sieving chromatography are VLPs to which allergens are bound. Both in terms of their size and their morphology, which comprises a phospholipid membrane covered with spikes, these VLPs closely resemble influenza virions (FIG. 5).

### Example 7: Production of Allergens or of Hypoallergens Carried by VLPs

The coupling of an allergen to a VLP considerably reduces its in vivo reactivity in IgEs from patients' serum.

However, the use of hypoallergens further reduces the reactivity of the IgEs and consequently the risks of anaphylactic reaction. The reduction in reactivity of a hypoallergenic form of the Der p2 allergen carried by VLPs is illustrated in FIG. 6.

### Example 8: VLP Production

The detailed method for producing the VLPs, up to their purification (as described in example 6), is illustrated in FIG. 7.

### Example 9: Immunogenic Power of the VLPs in Comparison With the Soluble Allergens

The presentation of an antigen in a highly ordered and repeating network normally brings about strong immune responses, whereas the same antigen presented as a monomer is non-immunogenic.

In order to compare the immune response with respect to the allergen when it is presented in the form of a highly ordered network, mice were immunized with the Der p2 allergen in soluble monomer form or in VLP-carried form. The titers of IgG against the Der p2 allergen were determined by ELISA.

### Protocol

The protocol is illustrated in FIG. 10.

Analyses after the sacrifice:
Weight loss and change in behavior
Pulmonary fuction (flexi Vent, plethysmography)
Serological response to the allergen (serum IgG, IgE, blot)
Basophil activation test
Histopathology and other serological results (for example: IgG isoforms) in the subsequent phases.

This study demonstrated that the VLPs coupled to the Der p2 allergen do not trigger bronchial hypereactivity in mice, contrary to soluble Der p2 (see FIG. 9, panel A). Furthermore, the VLPs bonded to Der p2 trigger a systemic Th1-type response with neutrophil activation (see FIG. 9, panels B and C).

### Example 10: Desensitization/Vaccination Using the VLPs

The key parameters of an effective vaccine are the following: rapid induction of a high antibody titer in the absence of adjuvants, and the absence of major side effects.

### Protocol

The protocol is illustrated in FIG. 11.

### LITERATURE REFERENCES

The references cited in the present application are the following:
1—Noon L. Prophylactic inoculation against hay fever Lancet 1911; 1:1572-3
2—Freeman J. Further observation on the treatment of hay fever by hypodermic inoculations of pollen vaccine. Lancet 1911; 2:814-817
3—Henmar et al. Clin Exp Immunol 2008; 153:316-323
4—Valenta et al. J Allergy Clin Immunol 2007; 119:826 830
5—Larche M. J Allergy Clin Immunol 2007; 119:906-909
6—Patel D. et al. J Allergy Clin Immunol 2013; 131:103-109
8—Chen et al. Allergy 2012; 67:609-621
9—Kundig et al. J Allergy Clin Immunol 2006; 117:1470-1476
10—Bachmann M F, Jennings G T Phil Trans R Soc B Biol SCI 2011; 366:2815-2822
11—Klimek et al. Am J Rhinol Allergy 2013; 27:206-212
12—Henmar et al. Clin Exp Immunol 2008; 153:316-323.
13—Jegerlehner et al. Eur J Immunol2002; 32:3305-3314.
14—D'aoust et al. Plant Biotech J 2008; 6:930-940
15—Garland S M et al. N Engl J Med 2007; 356:1928-1943
16—Paavonen et al. Lancet.2007; 369:2161-2170
17—Schmitz et al. J. Exp. Med. 2009; 206:1941-1955
18—Kundig et al. J Allerg Clin Immunol; 2006; 117:1470-1476
19—Cielens et al. FEBS Letters 2000; 482:261-264

1. A virus-like particle comprising:
an envelope consisting of a plasma membrane of which at least one portion is typical of lipid rafts; and
at least one type I or II transmembrane fusion protein anchored in said membrane, said fusion protein comprising the following fragments, successively:
b) a protein or a peptide of interest;
c) a coiled-coil domain or oligomerization sequence, which does not originate from a virus; and
d) a domain for anchoring in the plasma membrane, consisting of a transmembrane segment and a cytosolic segment, preferably a domain for anchoring in a plasma membrane of which at least one portion is typical of lipid rafts, fragments b) and c) being exposed at the surface of the virus-like particle.

2. The virus-like particle according to claim 1, wherein a linker is present between fragments b) and c), and/or between fragments c) and d).

3. The virus-like particle according to claim 1, wherein:
b) the protein or the peptide of interest is chosen from:
allergens and fragments thereof,
cell surface proteins and fragments thereof,
proteins and peptides that are accumulated in chronic or neurodegenerative diseases,
proteins and peptides involved in hypertension,
immunoglobulins and fragments thereof,
cytokines and fragments thereof, and
hormones and fragments thereof;
c) the coiled-coil domain is chosen from SEQ ID NO: 24, SEQ ID NO: 27, SEQ ID NO: 28, SEQ ID NO: 29, SEQ ID NO: 33 and SEQ ID NO: 30; and
d) the anchoring domain is chosen from the anchoring sequence of the H5N1 influenza virus H5 hemagglutinin (SEQ ID NO: 26) and the anchoring sequence of the PDLP1 protein (SEQ ID NO: 31).

4. The virus-like particle according to claim 1, wherein the protein or the peptide of interest is chosen from allergens and fragments thereof.

5. A type I or type II transmembrane fusion protein comprising the following fragments, successively:
a) optionally, a signal peptide;
b) a protein or a peptide of interest;
c) a coiled-coil domain or oligomerization sequence, which does not originate from a virus; and
d) a domain for anchoring in the plasma membrane, consisting of a transmembrane segment and a cytosolic segment, preferably a domain for anchoring in a plasma membrane of which at least one portion is typical of lipid rafts.

6. The transmembrane fusion protein according to claim 5, wherein the protein or the peptide of interest is chosen from allergens and fragments thereof.

7. A nucleic acid encoding a fusion protein as claimed in claim 5.

8. A host cell or a vector comprising at least one nucleic acid according to claim 7.

9. A method of treatment comprising a step of administering, to a patient in need thereof, a therapeutically effective amount of the virus-like particle according to claim 1.

10. A method of allergen immunotherapy comprising a step of administering, to a patient in need thereof, a therapeutically effective amount of the virus-like particle according to claim 1.

11. A method for producing virus-like particles according to claim 1, comprising the expression a nucleic acid in eukaryotic cells, wherein the nucleic acid encodes a type I or type II transmembrane fusion protein comprising the following fragments, successively:
a) optionally, a signal peptide;
b) a protein or a peptide of interest;
c) a coiled-coil domain or oligomerization sequence, which does not originate from a virus; and
d) a domain for anchoring in the plasma membrane, consisting of a transmembrane segment and a cytosolic segment, preferably a domain for anchoring in a plasma membrane of which at least one portion is typical of lipid rafts.

12. The method according to claim 11, characterized in that the eukaryotic cells are plant cells, and in that it comprises the following steps:
a) transformation of agrobacteria with an expression vector comprising the nucleic acid functionally linked to a strong promoter; and
b) transfection of the plant cells with the agrobacteria obtained in step a), said transfection comprising the following steps:
b1) culture of the plant cells, under aeroponic or hydroponic conditions, and under LED lighting, preferably for four to six weeks under hydroponic conditions,
b2) agroinfiltration of the plant cells obtained in b1) under vacuum, with the agrobacteria obtained in step a), preferably carried out under vacuum by Venturi effect,
b3) return to culture of the plant cells obtained in b2), typically for 3 to 6 days, in order to obtain the virus-like particles,
then extraction of the virus-like particles obtained and purification, in particular by enzymatic extraction.
